(19) United States
(12) Patent Application Publication
(10) Pub. No.: US 2023/0087959 A1
(43) Pub. Date: Mar. 23, 2023

(54) DEPTH GAUGE SYSTEM

(71) Applicant: Teleflex Life Sciences Limited, Valletta (MT)

(72) Inventor: William Joseph Drasler, Minnetonka, MN (US)

(73) Assignee: Teleflex Life Sciences Limited, Valletta (MT)

(21) Appl. No.: 17/930,812

(22) Filed: Sep. 9, 2022

Related U.S. Application Data
(60) Provisional application No. 63/245,315, filed on Sep. 17, 2021.

(51) Int. Cl. A61M 25/00

(52) U.S. Cl. CPC A61M 25/003; A61M 2025/0031; A61M 2025/0037

(57) ABSTRACT

Disclosed are a puncture sealing system and methods of locating a depth of an arteriotomy. The systems can include elongated catheters that are configured to identify the depth within a vessel so that the depth relative to a distal end of the catheter is known during or after a puncture sealing procedure.

### CROSS-REFERENCE TO RELATED APPLICATIONS

This application claims the benefit of and priority to, under 35 U.S.C. § 119(e) of U.S. Provisional Application No. 63/245,315 filed on Sep. 17, 2021, the entire content of which is incorporated herein by reference.

### TECHNICAL FIELD

The present application relates to a depth gauge system and method, and in particular, to a depth gauge catheter system utilized in surgical procedures.

### BACKGROUND

During the use of vascular closure systems after vascular interventions, it is often important to know the depth of an arteriotomy or tissue tract below the skin. In particular, the depth of the arteriotomy must be determined for procedures that form a large bore opening in the blood vessel such as a femoral artery, for example, that was used as an access site for a TAVR procedure or other large bore procedures. Typically, blood from the vessel can enter a catheter orifice located within the blood vessel near the arteriotomy site and give a blood leakage signal to a bleed port outside of a patient's body to signal the depth of the arteriotomy below the patient's skin puncture site.

Such depth gauge catheters have been utilized to determine the depth of the arteriotomy, however more accurate catheters are desired. Placing such depth gauge catheters with a diameter larger that the arteriotomy diameter or the tissue tract diameter can block blood communication from the vessel to the catheter orifice. Likewise, reducing the diameter of current depth gauge catheters allow further communication of blood flow from the artery to the orifice located within the tissue tract resulting in an inability to properly locate the arteriotomy site.

### SUMMARY

There is a need to provide a depth gauge catheter that will easily and accurately locate an arteriotomy site following an interventional procedure using a large bore catheter. An embodiment of the present disclosure includes a device configured to determine a depth of a puncture in a vessel relative to a skin surface of a patient. The device includes a flexible elongated body that extends along a central longitudinal axis. The flexible elongated body includes a proximal end and a distal end spaced from the proximal end along the central longitudinal axis. The flexible elongated body further includes a first channel that extends from the proximal end toward the distal end along the central longitudinal axis. The flexible elongated body further also includes a second channel adjacent to the first channel and that extends from the proximal end toward the distal end along the central longitudinal axis. The second channel includes a) a proximal portion, b) a distal portion c) a mandrel positioned along the distal portion, and d) an orifice extending though a side wall of the elongated body and having the mandrel disposed at an end of the distal portion and that is open to the mandrel e) the distal portion configured to form a shaft curve from the catheter orifice toward the proximal end along the central longitudinal axis f) the distal portion configured to provide a straight axial lumen for the mandrel that extends distally from the orifice to the end of the distal portion. The device further includes a movable shaft disposed along the second channel and that extends from the proximal portion to the distal portion. The moveable shaft is configured to actuate the mandrel such that the mandrel engages with the puncture.

A further embodiment of the present invention includes a device configured to determine a depth of a puncture in an vessel relative to a skin surface of a patient. The device includes a flexible elongated body that extends along a central longitudinal axis. The flexible elongated body includes a proximal end. The flexible elongated body further includes a distal end spaced from the proximal end along the central longitudinal axis. The flexible elongated body further includes an inner channel that extends from the proximal end toward the distal end along the central longitudinal axis. The inner channel including a) a proximal portion, b) a distal portion configured to curve from the distal end toward the proximal end along the central longitudinal axis, c) a mandrel positioned along the distal portion, and d) an orifice disposed at an end of the distal portion and that is open to the mandrel. The device further includes a guidewire disposed along the inner channel and that extends from the proximal portion to the distal portion. The guidewire is configured to actuate the mandrel such that the mandrel engages with the puncture.

### BRIEF DESCRIPTION OF THE DRAWINGS

The foregoing summary, as well as the following detailed description, will be better understood when read in conjunction with the appended drawings. The drawings show illustrative embodiments of the disclosure, in which there is shown in the drawings example embodiments for the purposes of illustration. It should be understood, however, that the application is not limited to the precise arrangements and instrumentalities shown.

FIG. 1A is a perspective view of a depth gauge catheter according to an embodiment of the present disclosure;

FIG. 1B is a cross-section of the depth gauge catheter taken along line A-A in FIG. 1;

FIG. 1C is a cross-section of a distal portion of the depth gauge catheter taken along line B-B in FIG. 1;

FIG. 1D is a cross-section of the depth gauge catheter taken along line B-B in FIG. 1;

FIG. 1E is a cross-section of a proximal portion of the depth gauge catheter taken along line B-B in FIG. 1;

FIG. 2A is a perspective view of a depth gauge catheter according to an embodiment of the present disclosure;

FIG. 2B is a cross-section of the depth gauge catheter taken along line C-C in FIG. 2A;

FIG. 2C is a cross-section of the depth gauge catheter taken along line E-E in FIG. 2A;

FIG. 3A is a perspective view of a flexible elongated body according to an embodiment of the present disclosure;

FIG. 3B is a cross section of the elongated body taken along line F-F in FIG. 3A;

FIG. 3C is a cross section of the elongated body taken along line G-G in FIG. 3A;

FIG. 3D is a schematic of the elongated body shown in FIG. 3A positioned within a vessel according to an embodiment of the present disclosure;

FIG. 3E is a schematic of the elongated body shown in FIG. 3A positioned within a vessel according to an embodiment of the present disclosure;

FIG. 4 is a perspective view of a depth gauge catheter shown in FIGS. 1A-3C with a vascular closure device in accordance with an embodiment of the present disclosure;

FIG. 5A is a perspective view of the vascular closure device shown in FIG. 4;

FIG. 5B is a perspective view of the vascular closure device shown in FIG. 5A with a portion of the housing removed;

FIG. 5C is a is a perspective view of a sealing device associated with the vascular closure device shown in FIGS. 5A and 5B;

FIG. 5D is a side sectional view of a distal portion of the vascular closure device shown in FIGS. 5A-5C;

FIG. 6A is a schematic showing the depth gauge catheter shown in FIGS. 1A-1C positioned such that the distal portion is disposed within a vessel proximate to a vessel puncture;

FIG. 6B is a schematic showing the depth gauge catheter shown in FIG. 4 being positioned to engage with the arteriotomy;

FIG. 6C is a schematic showing the access sheath shown in FIG. 7 being moved into the vessel; and

FIG. 6D is a schematic showing a sheath dilator removed from the access channel and the sheath body positioned such that a depth marking on the sheath body that corresponds to the at least one marking on the puncture locating dilator is visible above the surface of the skin.

### DETAILED DESCRIPTION OF ILLUSTRATIVE EMBODIMENTS

Certain terminology is used in the following description for convenience only and is not limiting. The words “right”, “left”, “lower” and “upper” designate directions in the drawings to which reference is made. The words “proximally” and “distally” refer to directions toward and away from, respectively, the individual operating the system. The terminology includes the above-listed words, derivatives thereof and words of similar import.

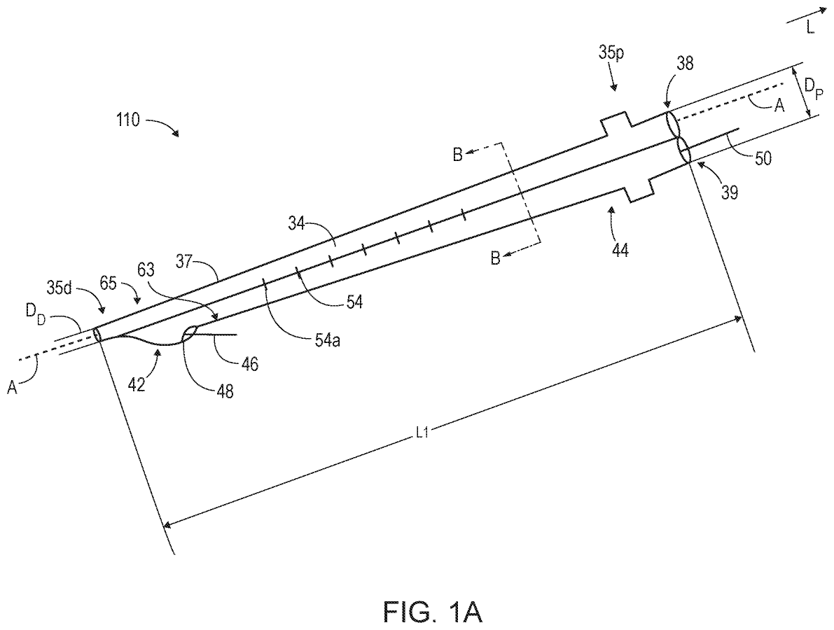

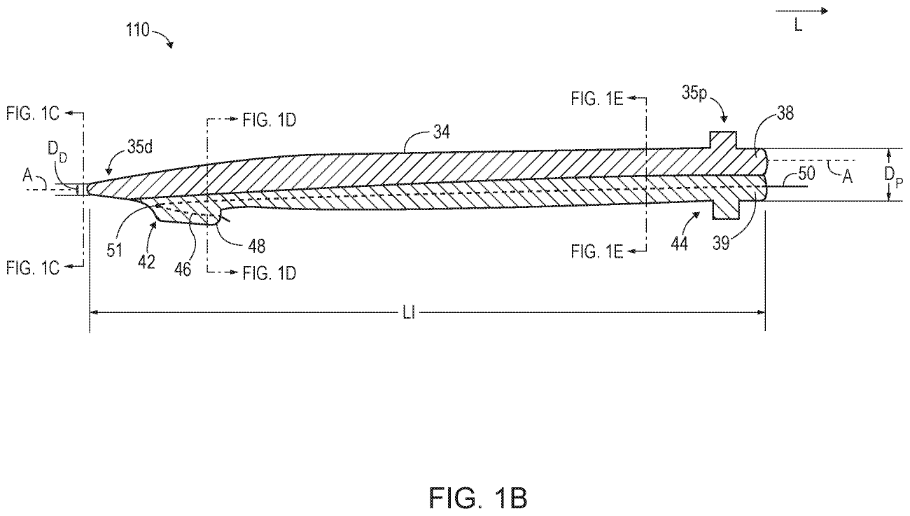

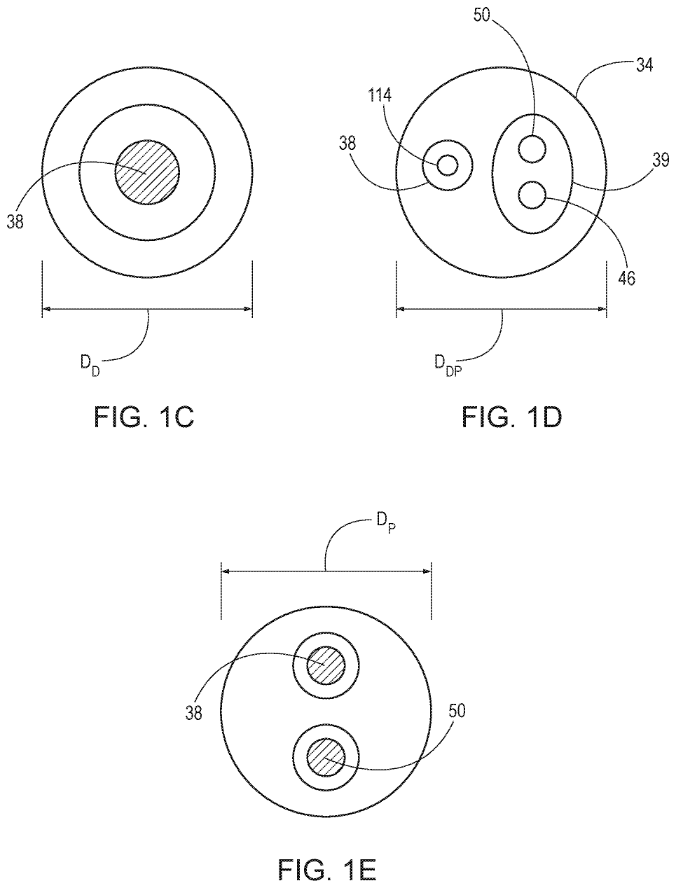

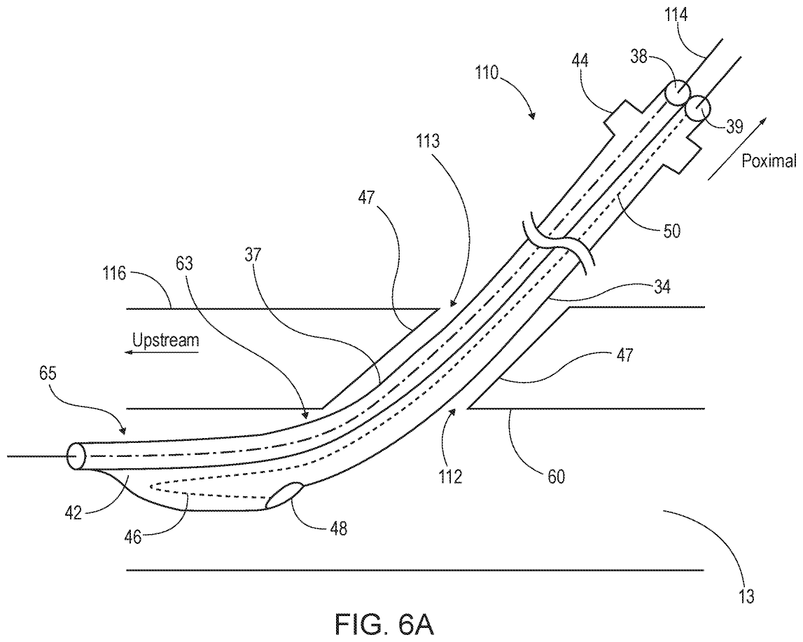

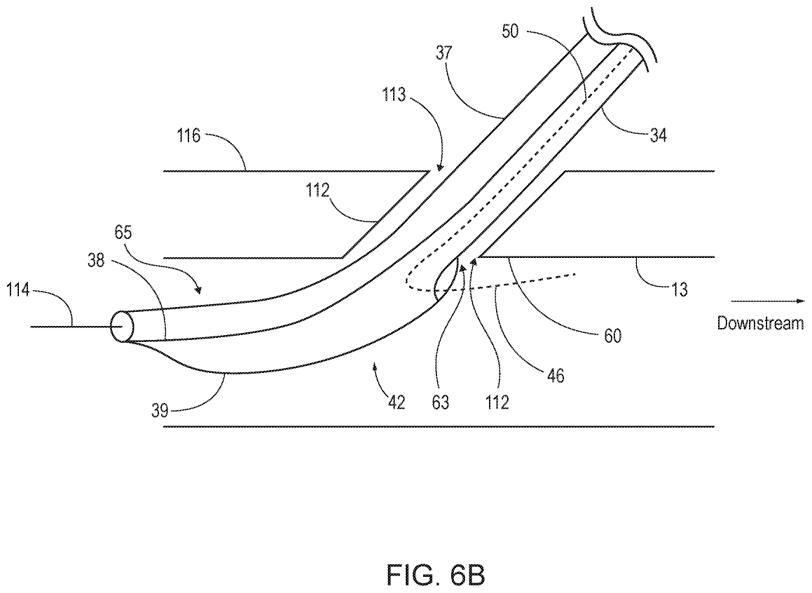

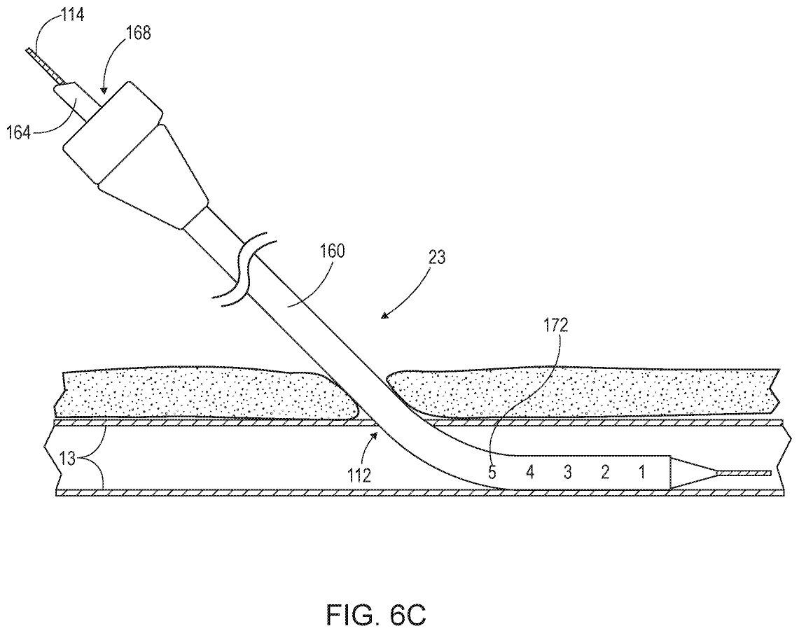

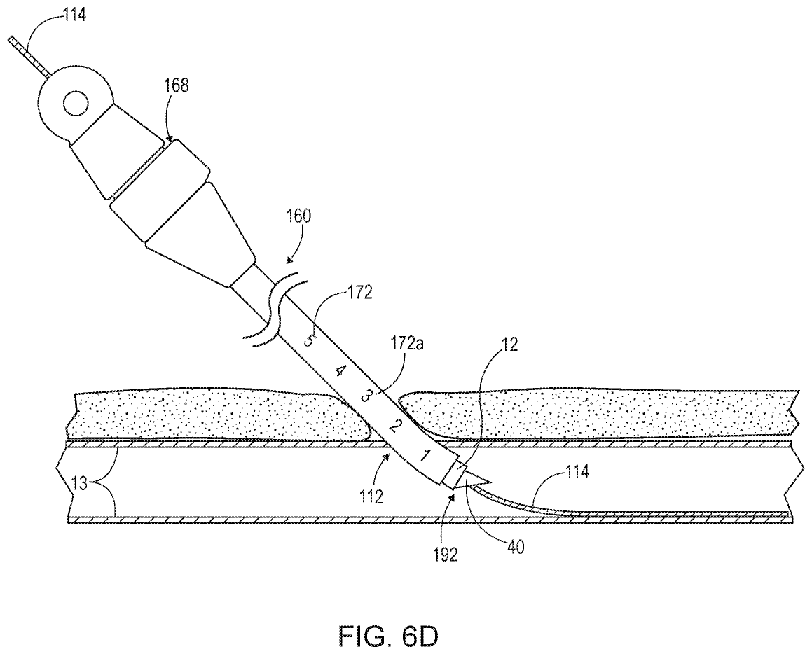

Referring to FIGS. 1A-1E and FIGS. 6A-6B, a depth gauge system in accordance with an embodiment of the invention can include a depth gauge catheter or depth gauge catheter 110 that is configured to determine the depth of an arteriotomy site 112 in a vessel of a patient from the skin puncture site 113 on the patient's skin 116. The depth gauge catheter 110 may be used to determine the depth of an arteriotomy during or along with an interventional cardiovascular procedure.

Typically, before an interventional cardiovascular procedure, a puncture may be made in the femoral artery. In one example, a vascular closure device composed of an absorbable anchor, a folding sealing plug, a suture and a downward locking member have been developed and may be used to seal these punctures. Such vascular closure devices are described in U.S. Patent Publication No. 2019/0110781, the entire contents of which are incorporated by reference here. However, before sealing can occur the depth at which the device needs to be inserted must be attained. Currently, the procedure is conducted with a depth gauge catheter of at least one distal port towards the distal end and one outlet opening at the proximal end.

Continuing with FIGS. 1A-1E, the depth gauge catheter 110 is an elongated catheter configured to determine the depth of site 112 in a vessel 13 (FIGS. 6A-6D). The depth gauge catheter 110 includes a flexible elongated body, or catheter body 34 that is elongated along a central longitudinal axis A in a first direction L. The first direction L is parallel to the central longitudinal axis A and may be referred to as a longitudinal direction in this disclosure. The catheter body 34 defines a proximal end 35p and a distal end 35d that is spaced from the proximal end 35p along the first direction L. The distal end 35d of the catheter 110 may be tapered to facilitate entry into the vessel.

The elongated body 34 is configured to be inserted into the arteriotomy site 112 to aid in locating an arteriotomy site. The flexible elongated body 34 further defines an outer surface 37, a first channel 38 that extends from the proximal end 35p toward the distal end 35d along the central longitudinal axis A1, and a second channel 39 that is adjacent to the first channel 38 and extends from the proximal end 35p toward the distal end 35d along the central longitudinal axis A. The first channel 38 and the second channel 39 each define an outer cross-sectional dimension that is substantially perpendicular to the central longitudinal axis A.

The first channel 38 is configured to be moved along a guide wire 114 (see FIGS. 6A-6C) toward the arteriotomy site 112 such that the depth gauge catheter 110 enters the vessel 13 through the arteriotomy site 112. As the depth gauge catheter 110 enters the vessel 13 the depth gauge catheter 110 dilates the arteriotomy site 112. The first channel 38 extends through the catheter body 34 along the first direction L from the distal end 35d to the proximal end 35p. The first channel 38 is configured to receive the guide wire 114 such that the depth gauge catheter 110 can be moved along the guide wire 114 toward the arteriotomy site 112.

The second channel 39 includes a distal portion 42 including an orifice 48 and a proximal portion 44. The distal portion 42 is configured to curve from the distal end 35d toward the proximal end 35p along the central longitudinal axis A at a location proximal to the catheter orifice 48 forming a shaft curve or shaft curve region 63. The shaft curve 63 can be thermally formed into the standard polymeric material used to form the catheter body. Alternately, the shaft curve 63 can be a region of the shaft located proximal to the catheter orifice 48 that has a reduced profile, for example, that provides the catheter body 34 with a greater flexibility and tendency to form a curve at a specific location along the catheter body. The distal portion 42 is configured to have a straight shaft 65 from the distal end 35d to the orifice 48 during actuation of the mandrel 46 out of orifice 48; the straight shaft 65 extends with a linear configuration to direct the mandrel 46 straight proximally to a position downstream of the arteriotomy site 112 and in line with the lumen of the blood vessel. The second channel 39 further includes a mandrel 46 and the orifice 48 that extends through a side wall of the second channel 39. The mandrel 46 is positioned inside of the second channel 39 toward the distal portion 42. The orifice 48 is disposed at an end of the distal portion 42 and is open to the mandrel 46. The mandrel 46 is configured to extend through the orifice 48 and retract in a proximal direction out of the catheter orifice 48. The mandrel 46 may also be retracted back into the distal portion 42 after the arteriotomy depth measurement has been made. The mandrel 46 forms a mandrel attachment 51 to the movable shaft 50 at an acute angle. The mandrel attachment 51 may be formed via a contiguous material forming both the mandrel 46 and the movable shaft 50; alternately, the mandrel 46 may be temporarily or permanently bonded, welded, or otherwise attached to the movable shaft 50 to form an acute angle of the mandrel attachment 51.

The mandrel 46 and the movable shaft 50 may be formed from a round or flat metal wire formed from stainless steel, Nitinol, or other metals or plastics used to form guidewires, for example, The mandrel 46 or the movable shaft 50 must support tension and compression and provide movement of the mandrel 46 within the catheter body 34 without kinking, breaking, and with low friction against the catheter body 34. The mandrel 46 must provide a low level of trauma to the blood vessel as it passes through the blood vessel and makes contact with the luminal wall of the blood vessel. The mandrel 46 can be formed from a flat wire having a rectangular cross sectional shape to provide a lower profile to the distal portion diameter by 10% and also provide a larger surface area for lower traumatic contact with the luminal surface of the blood vessel.

The depth gauge catheter 110 further includes a movable shaft 50 disposed along the second channel 39 and that extends from the proximal portion 44 to the distal portion 42. The movable shaft 50 further extends axially out from the proximal portion 44 of the second channel 39. The movable shaft 50 is configured to actuate the mandrel 46 such that the mandrel 46 engages with the arteriotomy site 112. The movable shaft 50 advances the mandrel 46 into the vessel 13 in a downstream direction to a location downstream of the arteriotomy site 112 such as the femoral artery arteriotomy site, for example, when used for coronary artery therapeutic interventions. When the catheter 110 is pulled under tension, the mandrel 46 engages with the luminal surface 60 of the blood vessel adjacent to the arteriotomy site 112 arteriotomy site 112 and prevents further proximal movement of the catheter 110.

The depth gauge catheter 110 can further include a plurality of depth markings 54 spaced from each other along the first direction L on the distal end 35d of the outer surface 37. The depth markings 54 can be used to visually note the depth or otherwise the location of the arteriotomy site 112 of the vessel 13 relative to the skin puncture site 113 when the depth gauge catheter 110 has been positioned within the vessel. In the illustrated embodiment, the plurality of depth markings 54 are etched into the outer surface 37. In one embodiment, the depth markings 54 are numbers aligned on the catheter body 34 along the central axis A. It should be appreciated, however, that the depth markings 54 can have other configurations as desired. For example, the depth markings can be configured as symbols as desired.

The depth markings 54 can be used to determine the depth of the arteriotomy site 112 from the skin puncture site 113. That is, a position of a first visible marking 54a of the plurality of depth markings 54 on the catheter 110 that is adjacent the patient's skin can be noted when the catheter is inserted. Therefore, the position of the arteriotomy site 112 can be known for the remainder of the procedure. The noted first depth marking 54a can be noted with a sticker that is placed directly on the patient's skin as desired. It should be appreciated, however, that the first depth marking 54a can be noted using other configurations as desired. For example, the first depth marking 54a can be noted with a tag, card, clip, etc. In an alternative embodiment, the depth markings of this embodiment can either be used alone or in combination with radiopaque markers.

Referring to FIGS. 1A-1E, the depth gauge catheter 110 is sized and shaped for a range of procedures. The elongated body 34 has a length L1 that extends from the proximal end 35p to the distal end 35d along the first direction L. In the illustrated embodiment, the length L1 is approximately between about 6 inches and 14 inches. In one example, the length L1 is about 10 inches. The flexible elongated body 34 includes a cross-sectional dimension D that is perpendicular to and intersects a central axis. As described above, the distal end 35d of the catheter 110 may be tapered to facilitate entry into the vessel. Thus, the proximal end 35p of the first channel 38 has a proximal diameter D_{P} that extends through and intersects the central axis A. The distal end 35d of the first channel 38 has a distal diameter D_{D} that extends through and intersects the central axis A and that is less than the proximal diameter D_{P} The distal portion of the catheter body 34 that contains both the movable shaft 50 and the mandrel 46 has a larger diameter D_{DP} than the distal diameter and has a cross-section as shown in FIG. 1D. The diameter of the mandrel 46 and the diameter of the movable shaft 50 can range from 0.014-0.035 inches. The guidewire typically used has a 0.035 inch diameter although a smaller diameter guidewire of 0.025 inches can alternately be used. In the illustrated embodiment, the proximal diameter D_{P} as shown in FIG. 1C may range from approximately 0.060-0.100 inches. Additionally, in the illustrated embodiment, the distal diameter D_{D} as shown in FIG. 1E may range from approximately 0.040-0.070 inches. The diameter of the distal portion, D_{DP}, as shown in FIG. 1D may range from approximately 0.060-0.100 inches.

The flexible elongated body 34 has a distal diameter D_{D} of the first channel 38 of approximately 0.040-0.055 inches to accommodate a −0.025-0.035 inch diameter guidewire. The diameter of the distal portion, D_{DP}, may be configured to accommodate a 0.025 inch diameter guidewire plus the mandrel 46 and the movable shaft 50 each with a diameter of 0.016 inches. Therefore, in one example, the diameter of the distal portion, D_{DP}, may be approximately 0.065 inches. An elongate lumen 118 of the second channel 39 has a major axis of approximately 0.056 inches and a minor axis of 0.032 inches to accommodate, for example, a 0.016 inch diameter movable shaft 50 and mandrel 46. Further, in one example, the first channel 38 and the second channel 39 comprise between 65% and 80% of the distal diameter D_{D} of the flexible elongated body. In another example, the first channel 38 and the second channel 39 comprises about 75% of the distal diameter D_{D} of the flexible elongated body 34. In a further example, the second channel 39 comprises up to about 10% of the distal diameter D_{D} of the flexible elongated body 34. The second channel 39 may comprise between about 3% and 8% of the distal diameter D_{D} of the flexible elongated body 34. The first channel 38 may also comprise between about 20% and 35% of the distal diameter D_{D} of the flexible elongated body 34. The first channel 38 may comprise between about 25% and 30% of the distal diameter D_{D} of the flexible elongated body 34. However, dimensions outside of these ranges are possible.

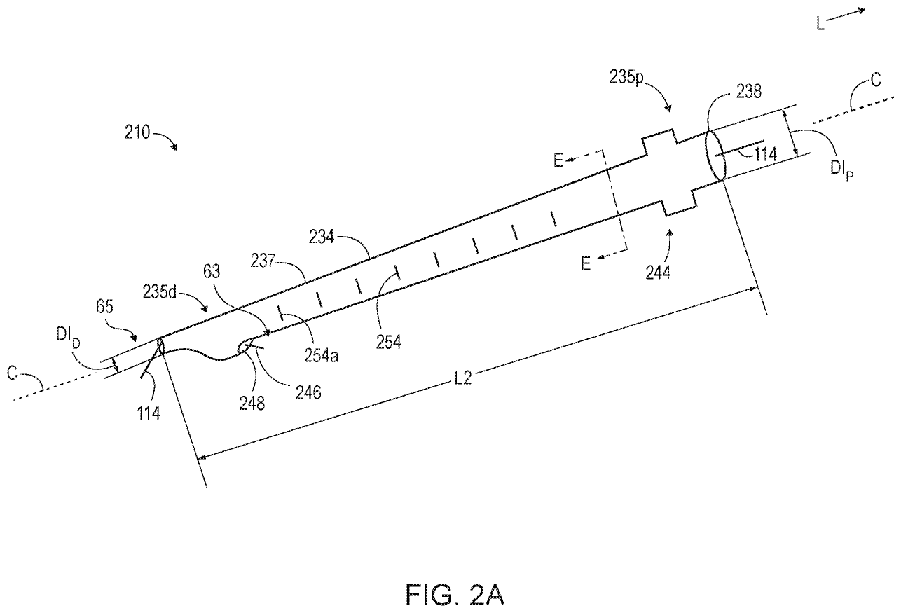

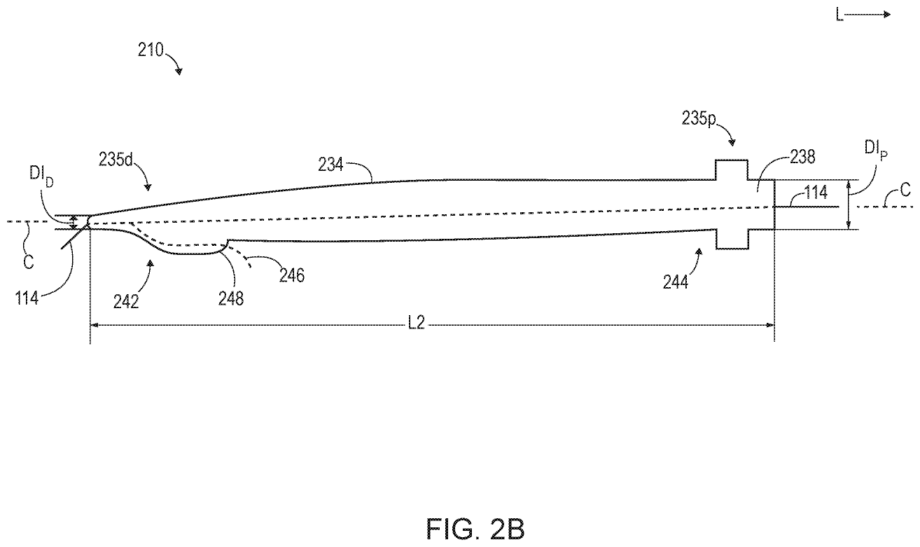

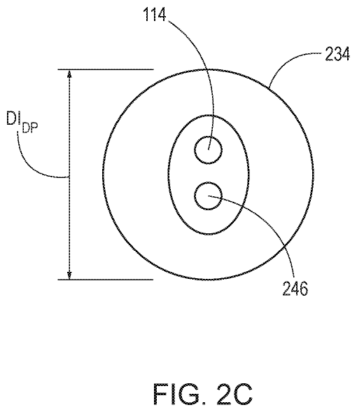

Referring to FIGS. 2A-2C, a depth gauge system in accordance with another embodiment of the invention can include a depth gauge catheter 210. The depth gauge catheter 210 is an elongated catheter configured to determine the depth of a arteriotomy site 112 in a vessel 13 (FIGS. 6A-6D). The depth gauge catheter 210 is similar to the depth gauge catheter 110 and common reference numbers are used to identify features that are common between the two described catheters. The depth gauge catheter 210 includes a flexible elongated body, or catheter body 234 that is elongated along a central longitudinal axis C in a first direction L. The first direction L is parallel to the central longitudinal axis C and may be referred to as a longitudinal direction in this disclosure. The catheter body 234 defines a proximal end 235p and a distal end 235d that is spaced from the proximal end 235p along the first direction L. The distal end 235d of the catheter 210 may be tapered to facilitate entry into the vessel.

The depth gauge catheter 210 may have similar dimensions to the depth gauge catheter 110. The catheter body 234 further defines an outer surface 237 and an inner channel 238 opposite the outer surface 237 that extends from the proximal end 235p toward the distal end 235d along the central longitudinal axis C. The inner channel 238 defines a cross-sectional dimension that is substantially perpendicular to the central longitudinal axis C.

The inner channel 238 is configured to be moved along a guide wire 114 (see FIG. 2B) toward the arteriotomy site 112 (not depicted) such that the depth gauge catheter 210 enters the vessel 13 (not depicted) through the arteriotomy site 112 (not depicted). The inner channel 238 extends through the catheter body 234 along the first direction L from the distal end 235d through to the proximal end 235p. The inner channel 238 is configured to receive the guide wire 114 such that the depth gauge catheter 210 can be moved along the guide wire 114 toward the arteriotomy site 112.

The inner channel 238 further includes a distal portion 242 and a proximal portion 244. The distal portion 242 is configured to form a shaft curve 63 from the distal end 235d toward the proximal end 235p along the central longitudinal axis C at a location proximal to the catheter orifice; the distal portion 242 has a linear and straight configuration forming a straight shaft 65 with the central longitudinal axis C near the distal end 235d to direct the mandrel 246 straight downstream within the blood vessel and past the arteriotomy site 112 prior to withdrawing the catheter proximally under tension. The inner channel 238 further includes a mandrel 246 and an orifice 248. The mandrel 246 and the orifice 248 may be similar in structure and function to the mandrel 146 and the orifice 148 of the depth gauge catheter 110. The mandrel 246 is positioned inside of the inner channel 238 toward the distal portion 242. The orifice 248 is disposed at an end of the distal portion 242 and is open to the mandrel 246. The mandrel 246 is configured to extend through the orifice 248 and retract back into the distal portion 242 of the inner channel 238 when the mandrel 246 is actuated inside the vessel 13.

The guide wire 114 further extends axially out from the proximal portion 244 of the inner channel 238. The guide wire 114 is configured to actuate the mandrel 246 such that the mandrel 246 engages with the arteriotomy site 112 as described for the embodiment of FIGS. 1A-1E. The guide wire 114 is configured to advance the mandrel 246 into the vessel 13 in a downstream direction to a location downstream of the arteriotomy site 112 such as for an arteriotomy site made within the femoral artery for providing a therapeutic treatment access site for a coronary procedure, for example. When the catheter 210 is pulled under tension, the mandrel 246 engages with the arteriotomy site 112 and prevents further proximal movement of the catheter 210.

The depth gauge catheter 210 can further include a plurality of depth markings 254 spaced from each other along the first direction L on the distal end 235d of the outer surface 237. The depth markings 254 can be used to visually note the depth or otherwise the location of the arteriotomy site 112 of the vessel 13 when the depth gauge catheter 210 has been positioned within the vessel. In the illustrated embodiment, the plurality of depth markings 254 are etched into the outer surface 237. The plurality of depth markings 254 are similar in structure and function to the plurality of depth markings 54.

Referring to FIGS. 2A-2C, the depth gauge catheter 210 is sized and shaped for a range of procedures. The catheter body 234 has a length L2 that extends from the proximal end 235p to the distal end 235d along the first direction L. In the illustrated embodiment, the length L2 is approximately between about 8 inches and 14 inches. In one example, the length L2 is about 10 inches. The flexible elongated body 234 includes a cross-sectional diameter DI. The cross-sectional diameter DI may be similar to the cross-sectional diameter D of the flexible elongated body 34. As described above, the distal end 235d of the catheter 210 may be tapered to facilitate entry into the vessel. Thus, the proximal end 235p of the inner channel 238 has a proximal diameter DI_{P} that extends through and intersects the central axis C. The distal end 235d of the inner channel 238 has a distal diameter DI_{D} that extends through and intersects the central axis C and that is less than the proximal diameter DI_{P}. In the illustrated embodiment, the proximal diameter DI_{P} may range from approximately 0.040-0.055 inches. Additionally, in the illustrated embodiment, the distal diameter DI_{D} may range from approximately 0.065-0.085 inches.

The flexible elongated body 234 includes a radius R2 measured from the outer surface 237 to the center of the flexible elongated body 234. The radius R2 may have similar ranges to the radius R1 of the depth gauge catheter 110. However, dimensions outside of these ranges are possible.

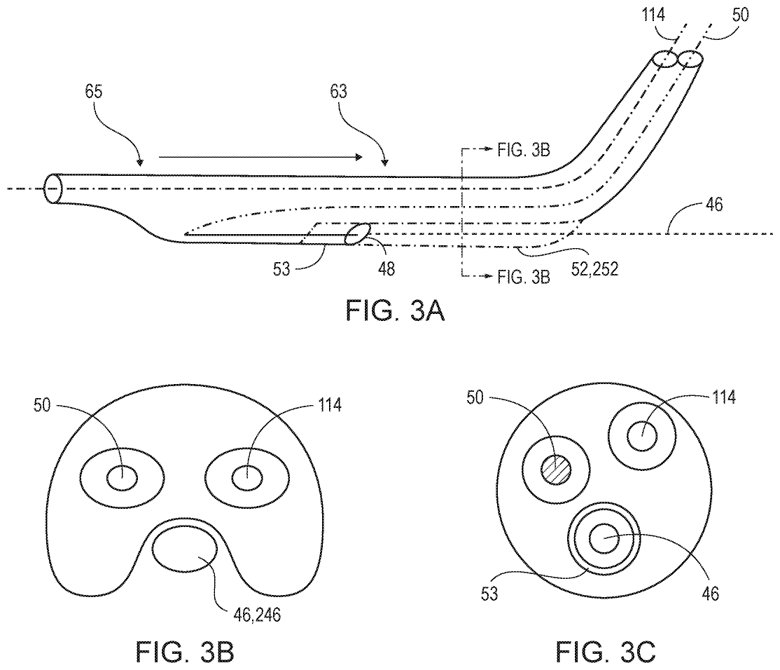

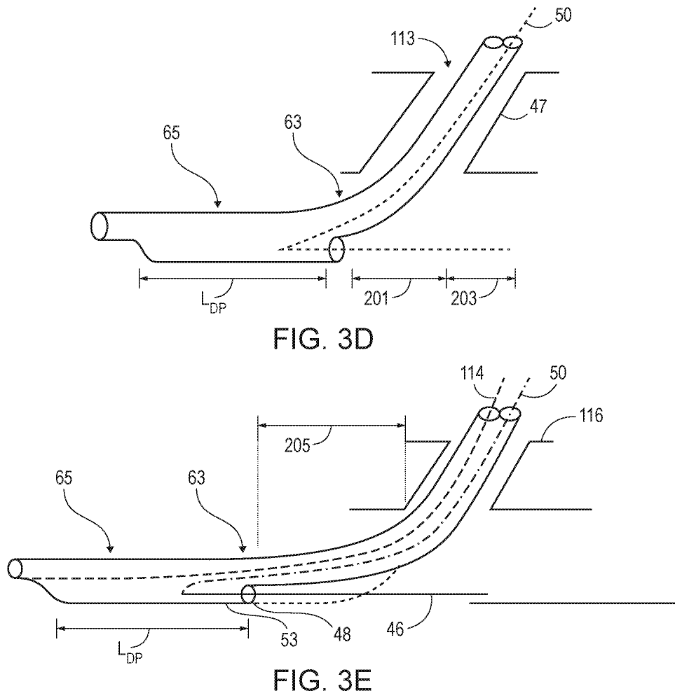

Referring to FIGS. 3A-3C, the flexible elongated body 34, 234 may include a cradle 52, 252 disposed along the catheter body 34, 234 proximal to the orifice 48, 248 and extending for a distance of zero to 60 mm. The cradle 52, 252 is configured to provide a cross-sectional space for the mandrel 46, 246 to pass out of the orifice 48, 248 in a downstream direction in the vessel 13 without interfering with the vessel wall. The distal portion 42, 242 further may include an alignment member 53, 253 distal to the orifice 48, 248. The alignment member 53, 253 is configured to direct the mandrel 46, 246 in a downstream direction within the vessel 13 and prevent the mandrel 46, 246 from extending proximally into the arteriotomy site 112 when the catheter 110, 210 is placed under tension. The alignment member 53, 253 can be a cylindrical tube or a cylindrical lumen that extends distally from the orifice 48, 248 for a distance of approximately 2-6 mm to direct the mandrel 46 in line with the distal portion as the mandrel 46, 246 exits the orifice 48, 248. The alignment member 53, 253 can be formed from the standard thermoplastic that forms the catheter body or can be a plastic tube inserted into the catheter orifice and extending into the distal portion. Alternately, the alignment member 53, 253 can be a polymeric tab or polymeric protrusion extending into the lumen of the second channel that makes contact with a surface of the mandrel 46, 246 to provide a mandrel exit from the orifice 48, 248 that is in alignment with the distal portion.

FIGS. 3D and 3E show the depth gauge catheter of the present invention located within a blood vessel. In FIG. 3D, the catheter has been placed using fluoroscopy or other visualization modality such that the catheter orifice 48, 248 is located just upstream of the arteriotomy site 112. A radiopaque marker band or other visualization marker (not shown) located near the orifice 48, 248 can assist in placing the catheter orifice near the arteriotomy site 112. In this configuration, a distal portion length of approximately 18 mm (range 12-25 mm) will accommodate the mandrel 46, 246 contained within the catheter during delivery such that the mandrel 46, 246 subtends the arteriotomy length 201 (approximately 8 mm for a femoral artery arteriotomy) and overlaps downstream of the arteriotomy by approximately 10 mm. The catheter can form a shaft curve 63 in the catheter body located proximal to the orifice 48, 248 as the catheter body extends into the tissue tract 47, 247. The catheter body forms a straight shaft 65 extending from the catheter orifice 48, 248 to the distal end. An alignment member and cradle may not be necessary if the catheter orifice 48, 248 is located near and adjacent to the arteriotomy site 112. The mandrel length for this configuration is approximately 18 mm.

FIG. 3E shows the catheter being placed into the blood vessel without the need or use of fluoroscopy or other visualization modality. The catheter orifice 48, 248 is advanced into the blood vessel to a greater distance in order to provide a safety margin that ensures that the orifice is located upstream of the arteriotomy site. This margin distance 205 from orifice 48, 248 to arteriotomy site 112 can be between approximately 20 mm and 50 mm. In one embodiment, the margin distance 205 may be 35 mm. For this configuration a distal portion length, L_{DP}, of approximately 55 mm (range 40-80 mm) can accommodate a cylindrical alignment member (length 2-6 mm), provide for a 35 mm margin distance, subtend a femoral artery arteriotomy of approximately 8 mm, and provide an overlap length 203 of approximately 10 mm. A shaft curve or shaft curve region 65 can be formed in the catheter body at a location proximal to the catheter orifice 48, 248. The catheter body forms a straight shaft 65 extending from the catheter orifice to the distal end. A cradle that is formed into the catheter body 34, 234 proximal to the catheter orifice 48, 248 for a distance of approximately 35 mm, for example, can direct the mandrel proximally downstream from the orifice without mandrel impingement upon potential plaque within the artery to a location downstream of the arteriotomy site. The mandrel length for this configuration is approximately 55 mm.

In one embodiment, the depth gauge system may deliver the catheter with the orifice 48, 248 upstream of the arteriotomy site. The movable shaft 50 is withdrawn to actuate the mandrel 46, 246 out of the orifice. The catheter is then withdrawn under tension to place the mandrel against the luminal surface of the blood vessel with the orifice 48, 248 adjacent to the arteriotomy site 112. The catheter depth markings 54, 254 are recorded by the operator to measure the distance from the arteriotomy site to the skin puncture site. The movable shaft 50 is then advanced distally under compression to retract the mandrel 46, 246 back into the catheter. The catheter 110 can then be removed from the patient or can otherwise be retained as part of a closure device which is then properly activated with knowledge of the depth of the arteriotomy site.

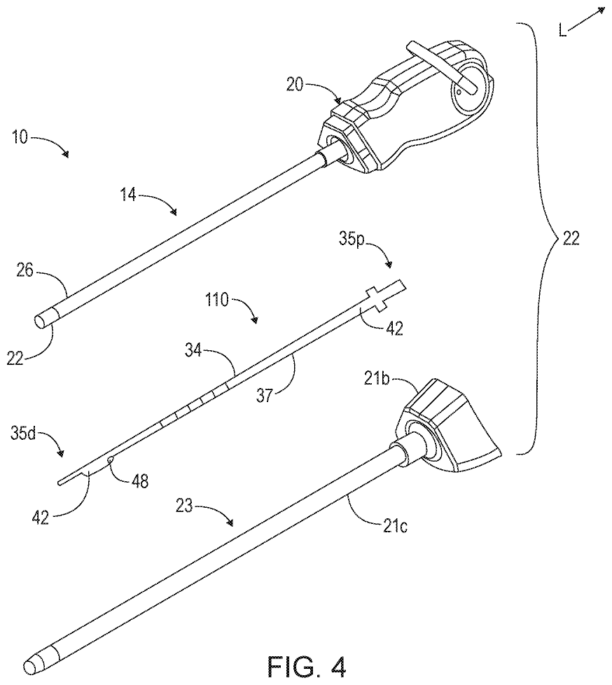

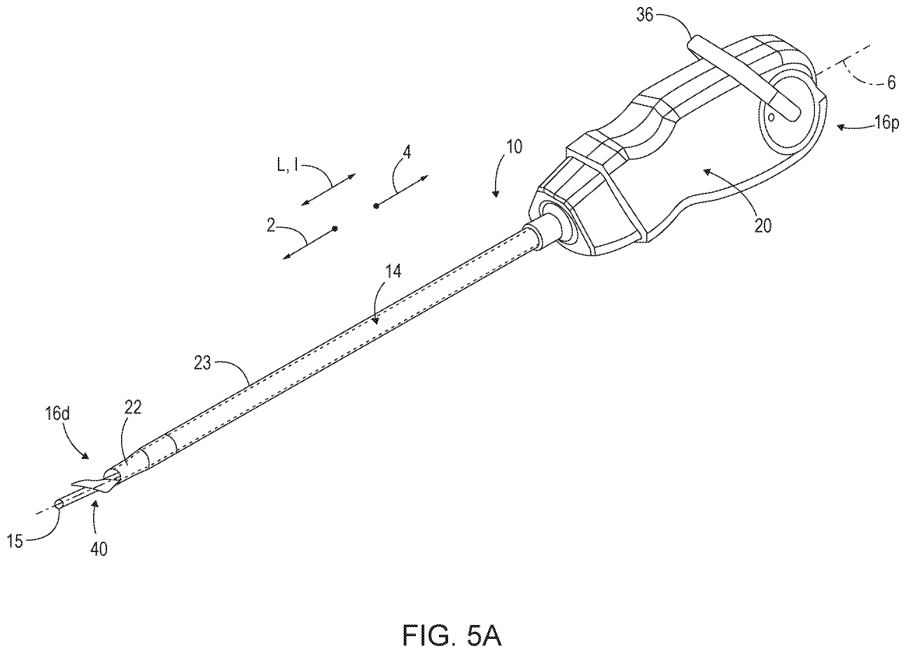

Referring to FIG. 4, in the illustrated embodiment, the depth gauge catheter 110 described herein is used in connection with a vascular closure system 10 to determine the depth of the arteriotomy site 112 during vascular closure procedures. In alternative embodiments, the depth gauge catheter 110 described herein may be used generally for depth determination in a vessel 13. As shown in FIG. 4, the puncture sealing system can further include an access sheath 23 that is also configured to be moved along the guide wire 114 toward the arteriotomy site 112 and into the vessel 13 so as to further dilate the arteriotomy site 112 and subsequently provide access to the vessel 13. The access sheath 23 can then receive a sealing device that is configured to seal the arteriotomy site 112. It should be appreciated, however, that the system can include additional dilators that have cross-sectional diameters that are different (e.g. greater) than the diameter of the depth gauge catheter 110 but less than that of the access sheath 23 so that the arteriotomy site 112 can be gradually dilated and prepared for the access sheath 23.

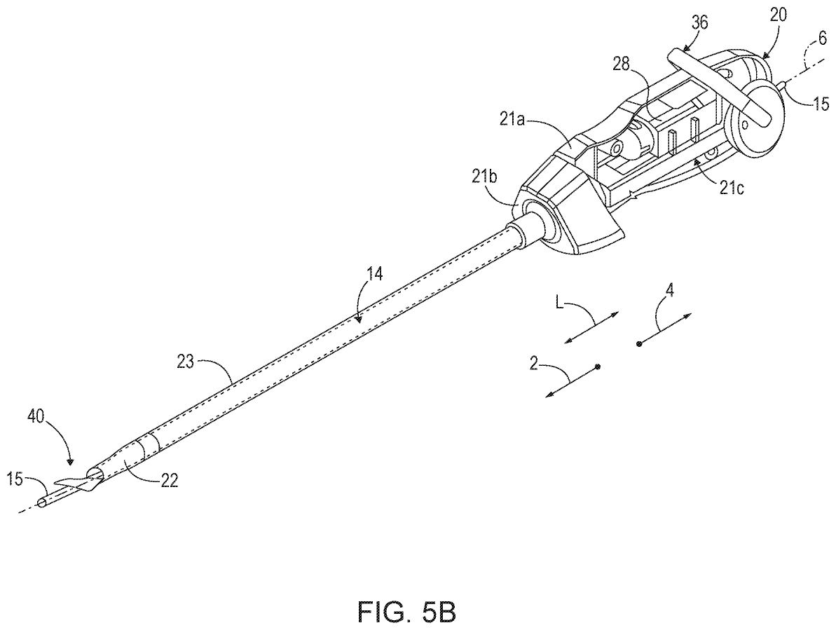

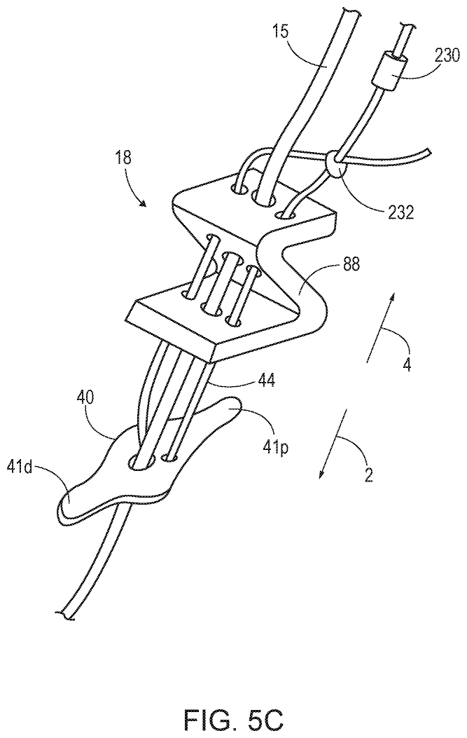

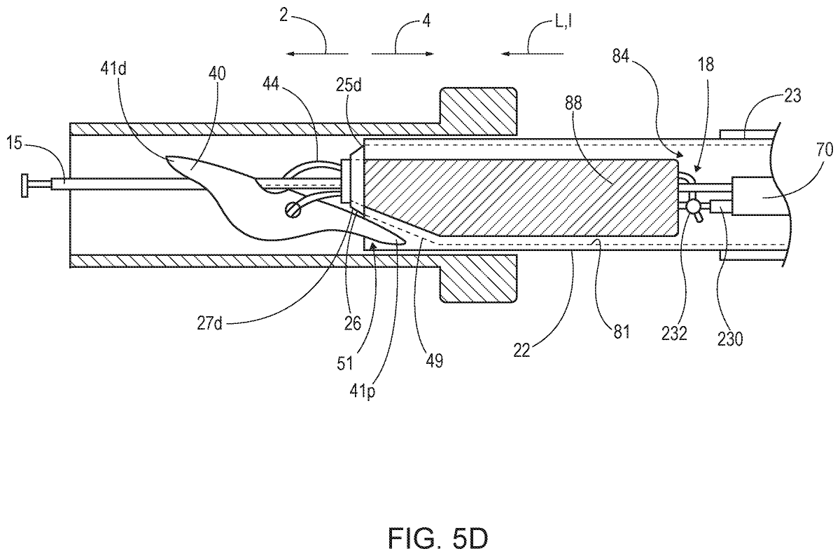

Continuing with FIGS. 4-5D, the vascular closure system 10 includes a closure device 12 that is configured to seal a puncture in a vessel wall. The depth gauge catheter 110 is configured to determine a depth of the arteriotomy site 112 to facilitate placement of the closure device 12 into the desired position within a skin puncture site of a vessel wall following a surgical procedure. The closure device 12 includes a deployment assembly 14 and an access sheath 23. The access sheath 23 can be inserted into the vessel and the deployment assembly 14 can be inserted into the access sheath 23 to position a sealing unit 18 (FIG. 5C) into the vessel.

Referring to FIGS. 5A and 5B, a vascular closure device 12 includes a sealing unit 18 at least partially disposed within a deployment assembly 14. The vascular closure device 12 can be configured such that after a distal portion of deployment assembly 14 is inserted through a arteriotomy site 112 of the vessel 13, the sealing unit 18 is deployed to thereby seal or otherwise close the arteriotomy site 112 of the vessel 13. The deployment assembly 14 is configured to control orientation of a toggle 40 of the sealing unit 18 in an easier and more efficient manner during deployment of the sealing unit 18. Furthermore, the deployment assembly 14 is configured to reduce forces required to deploy the sealing unit 18 and seal the puncture.

In accordance with the illustrated embodiment, the deployment assembly 14 includes a release component 22 that restrains the toggle 40, a delivery component 26 (See FIG. 5D) that contains at least a portion of the toggle 40 and a suture 43 of the sealing unit 18, a guide member 15, and one or more actuators 36 coupled to the release component 22. The deployment assembly 14 may also include a tamper 70, in the form a tube, that extends along the suture 43 and is located in a proximal direction relative to the locking member 230 (See FIG. 5D). The guide member 15 extends through the sealing unit 18 and is configured to receive a guide wire as will be discussed below. In another example, the deployment assembly 14 can be configured so that the guide wire 114 extends along the side of the toggle 40. The release component 22 is operatively associated with the suture 43 such that actuation of the actuator 36 causes the release component 22 to 1) release the toggle 40, and 2) apply tension to the suture 43, which urges the toggle 40 against the delivery component 26 and orients the toggle 40 in the sealing position. The guide member 15 is configured to be removed from at least the sealing unit 18 prior to the sealing unit 18 sealing the puncture.

Turning to FIG. 5C, the sealing unit 18 includes the toggle 40 connected to the suture 43, a plug 88 coupled to the suture 43 and spaced from the toggle 40 in a proximal direction 4, and a locking member 230 proximal to the plug 88. The toggle 40 includes a distal end 45 d and a proximal end 41 p opposite to the proximal end 41 p, and a plurality of apertures (not numbered) extending therethrough. The suture 43 extends through the apertures as illustrated such that an end of the suture 43 is formed into a slidable knot 232. The knot 232 is slidable along the suture 43 between the plug 88 and the locking member 230. In an implanted state, the toggle 40 is adjacent to an inner surface of the vessel and the locking member 230 squeezes the toggle 40 and the plug 88 against the vessel to seal the puncture.

The sealing unit 18 is formed with materials suitable for surgical procedures such as any biocompatible material. It should be appreciated, however, that the toggle 40 can be made of other materials and can have other configurations so long as it can be seated inside the vessel against the vessel wall. The plug 88 can comprise a strip of compressible, resorbable, collagen foam and can be made of a fibrous collagen mix of insoluble and soluble collagen that is cross linked for strength. It should be appreciated, however, that the plug member 88 can have any configuration as desired and can be made from any material as desired. The suture 43 can be any elongate member, such as, for example a filament, thread, or braid.

Now referring to FIGS. 6A-6D, the guide wire 114 can be inserted through the arteriotomy site 112 and into the vessel 13 such that a portion of the guide wire 114 protrudes from the vessel. Once the guide wire 114 is positioned, a proximal end of the guide wire 114 can be inserted into the distal end of the first channel 38 of the depth gauge catheter 110. As shown in FIG. 6A, the depth gauge catheter 110 can then be moved along the guide wire 114 until the distal end of the depth gauge catheter 110 is directed upstream of the arteriotomy site 112 within the vessel 13. After the distal region of the catheter 110 is located within the vessel 13 the movable shaft is placed under tension to advance the mandrel 46 in a downstream direction in the vessel 13. The alignment member can direct the mandrel 46 in a downstream direction within a femoral artery, for example, parallel to the distal portion of the catheter body. The cradle can ensure that potential plaque located within the blood vessel lumen near the catheter orifice cannot interfere with the mandrel movement parallel with the distal portion downstream of the arteriotomy site prior to retraction of the catheter under tension. As shown in FIG. 6B, the catheter 110 is then pulled under tension partially out of the vessel 13 to locate the mandrel 46 against the blood vessel wall downstream of and adjacent to the arteriotomy site 112 such that the orifice 48 is located adjacent to the arteriotomy site 112. The mandrel 46 engages the arteriotomy site 112 and prevents further proximal movement of the entire depth gauge catheter 110 in order to assist in the identification of the depth of the arteriotomy site 112.

Continuing with FIG. 6B, after the mandrel 46 engages the arteriotomy site 112, a first visible marking 54a of the catheter 110 can be noted. That is, the first visible marking 54a that is adjacent the patient's skin can be noted. Once the depth of the arteriotomy site 112 has been recorded on the catheter 110, the mandrel is positioned back within the depth gauge catheter 110 by placing the movable shaft 50 under compression and the catheter 110 is removed from vessel 13. It should be appreciated, that in some embodiments, the depth gauge catheter 110 can be positioned over the guide wire 114 prior to the guide wire 114 being inserted into the vessel 13.

Referring to FIG. 6C, the depth gauge catheter 110 may be utilized with an access sheath 23 that is also configured to be moved along the guide wire 114 toward the arteriotomy site 112 and into the vessel 13 so as to further dilate the arteriotomy site 112 and subsequently provide access to the vessel 13. The access sheath 23 can then receive a vascular closure device 12 that is configured to seal the arteriotomy site 112. It should be appreciated, however, that the system can include additional sequentially sized dilators that have cross-sectional diameters that are different (e.g. greater) than the diameter of the depth gauge catheter 110 but less than that of the access sheath 23 so that the arteriotomy site 112 can be gradually dilated and prepared for the access sheath 23. Both the depth gauge catheter 110 and the access sheath 23 may include respective depth markings that are configured to aid in locating the arteriotomy site 112.

Continuing with FIG. 6C, after the depth gauge catheter 110 has been removed from the guide wire 114 and any subsequent dilators have been removed, the access sheath 23 can be moved along the guide wire 114 toward the arteriotomy site 112 such that the distal end of the access sheath 23 enters the vessel 13 through the arteriotomy site 112. In particular, the proximal end of the guide wire 114 is inserted into the distal end of a sheath dilator 164. And then the sheath body 160 and sheath dilator 164 can be moved together along the guide wire 114 toward the arteriotomy site 112. Once inserted, the sheath dilator 164 can be pulled proximally such that the sheath dilator 164 is removed from the access channel 168.

After the sheath dilator 164 has been removed, a vascular closure procedure can be performed through the access channel 168. Therefore, a closure device 12 can be moved into the access channel 168 until a distal portion 192 (e.g. at least a portion of the toggle 40) of the closure device 12 is distal to the distal end of the sheath body 160. As shown in FIG. 6D the access sheath 23 can then be moved such that a first visible marking 172a of the sheath body 160 that is visible adjacent the patient's skin corresponds with the noted first visible marking 54a of the depth gauge catheter 110. It should be appreciated, that the closure device 12 can be moved into the access channel 168 either prior to or after the positioning of the access sheath 23 such that the first visible marking 172a corresponds to the noted marking 54a. When the access sheath 23 is properly positioned, the closure device 12 will be positioned such that the sealing procedure can be completed. It should be appreciated, that while in the illustrated embodiment, the depth markings 172 are on the sheath body 160, in some embodiments, the depth markings can be on the closure device 12, as desired. Furthermore, it should be appreciated, that in such embodiments, the access sheath 23 can be pulled completely out of the vessel 13 when the closure device 12 is properly positioned.

While the foregoing description and drawings represent the preferred embodiment of the present invention, it will be understood that various additions, modifications, combinations and/or substitutions may be made therein without departing from the spirit and scope of the present disclosure as defined in the accompanying claims. In particular, it will be clear to those skilled in the art that the present disclosure may be embodied in other specific forms, structures, arrangements, proportions, and with other elements, materials, and components, without departing from the spirit or essential characteristics thereof. One skilled in the art will appreciate that the present disclosure may be used with many modifications of structure, arrangement, proportions, materials, and components, which are particularly adapted to specific environments and operative requirements without departing from the principles of the present disclosure. In addition, features described herein may be used singularly or in combination with other features. For example, features described in connection with one component may be used and/or interchanged with features described in another component. The presently disclosed embodiment is therefore to be considered in all respects as illustrative and not restrictive, the scope of the present disclosure being indicated by the appended claims, and not limited to the foregoing description. It will be appreciated by those skilled in the art that various modifications and alterations of the present disclosure can be made without departing from the broad scope of the appended claims. Some of these have been discussed above and others will be apparent to those skilled in the art.

What is claimed:

1. A device configured to determine a depth of a puncture in a vessel relative to a skin surface of a patient, the device comprising:
a flexible elongated body that extends along a central longitudinal axis, the flexible elongated body having
a proximal end,
a distal end spaced from the proximal end along the central longitudinal axis,
a first channel that extends from the proximal end toward the distal end along the central longitudinal axis,
a second channel adjacent to the first channel and that extends from the proximal end toward the distal end along the central longitudinal axis, the second channel including a) a proximal portion, and b) a distal portion opposite the proximal portion,
an orifice disposed at an end of the distal portion and that is open to the second channel, and
a mandrel positioned along the distal portion and that is in communication with the orifice; and
a movable shaft disposed inside the second channel and that extends from the proximal portion to the distal portion;
wherein the moveable shaft is configured to actuate the mandrel between a first position where the mandrel is disposed inside the second channel and a second position where a distal end of the mandrel projects from the orifice.

2. The device of claim 1, wherein the movable shaft advances the mandrel into the vessel in a downstream direction to a location downstream of the puncture.

3. The device of claim 2, wherein the mandrel engages with the puncture and prevents further proximal movement of the device when the device is pulled under tension.

4. The device of claim 3, wherein the distal portion further includes a cradle proximal to the orifice, the cradle configured to provide a cross-sectional space for the mandrel to pass out of the orifice in a downstream direction without interfering with the vessel.

5. The device of claim 4, wherein the distal portion further includes an alignment member that extends distally from the orifice, the alignment member configured to direct the mandrel in a downstream direction within the vessel and prevent the mandrel from extending proximally into the puncture when the movable shaft is placed under tension.

6. The device of claim 3 wherein said distal portion is configured to form a straight shaft and is configured to be in line with a blood vessel lumen.

7. The device of claim 3 wherein said catheter body has a shaft curve at a location proximal to said orifice.

8. The device of claim 1, wherein the flexible elongated body includes a plurality of markings spaced apart along a longitudinal direction that is parallel to the central longitudinal axis.

9. The device of claim 8, wherein the plurality of markings are etched into an outer surface of the flexible elongated body.

10. The device of claim 1, wherein the flexible elongated body has a tapered portion that defines the distal end and a linear portion that extends from the tapered portion to the proximal end, wherein the tapered portion tapers toward the central longitudinal axis.

11. The device of claim 1, wherein the first channel and the second channel comprise between 65% and 80% of a distal diameter of the flexible elongated body.

12. The device of claim 1, wherein at least a part of the distal portion is offset with respect to the proximal portion.

13. The device of claim 1, wherein at least a part of the distal portion is curved.

14. The device of claim 1, wherein the distal portion is curved from the distal end toward the proximal end along the central longitudinal axis.

15. A device configured to determine a depth of a puncture in an vessel relative to a skin surface of a patient, the device comprising:
a flexible elongated body that extends along a central longitudinal axis, the flexible elongated body having:
a proximal end,
a distal end spaced from the proximal end along the central longitudinal axis,
an inner channel that extends from the proximal end toward the distal end along the central longitudinal axis, the inner channel including a) a proximal portion, and b) a distal portion that curves from the distal end toward the proximal end along the central longitudinal axis,
an orifice disposed at an end of the distal portion and that is open to the inner channel, and a mandrel positioned along the distal portion and that is in communication with the orifice;
wherein the mandrel is actuatable between a first position where the mandrel is disposed inside the inner channel and a second position where a distal end of the mandrel projects from the orifice.

16. The device of claim 13, wherein the mandrel is advanced into the vessel in a downstream direction to a location downstream of the puncture.

17. The device of claim 14, wherein the mandrel engages with the puncture and prevents further proximal movement of the device when the device is pulled under tension.

18. The device of claim 15, wherein the distal portion further includes a cradle proximal to the orifice, the cradle configured to provide a cross-sectional space for the mandrel to pass out of the orifice in a downstream direction without interfering with the vessel.

19. The device of claim 16, wherein the distal portion further includes a tab proximal to the orifice, the tab configured to direct the mandrel in a downstream direction within the vessel and prevent the mandrel from extending proximally into the puncture.

20. The device of claim 13, wherein the flexible elongated body includes a plurality of markings spaced apart along a longitudinal direction that is parallel to the central longitudinal axis.

21. The device of claim 18, wherein the plurality of markings are etched into an outer surface of the flexible elongated body.

22. The device of claim 13, wherein the flexible elongated body has a tapered portion that defines the distal end and a linear portion that extends from the tapered portion to the proximal end, wherein the tapered portion tapers toward the central longitudinal axis.

23. The device of claim 13, wherein inner channel comprises between 65% and 80% of a distal diameter of the flexible elongated body.
